(19) United States
(12) Patent Application Publication
(10) Pub. No.: US 2020/0325085 A1
(43) Pub. Date: Oct. 15, 2020

(54) Linear Alpha Olefin Isomerization Using an Ebullated Bed Reactor

(71) Applicant: ExxonMobil Chemical Patents Inc., Baytown, TX (US)

(72) Inventors: Anatoly I. Kramer, Baytown, TX (US); Renyuan Yu, Humble, TX (US); Elizabeth G. Mahoney, Houston, TX (US); Paul F. Keusenkothen, Houston, TX (US)

(21) Appl. No.: 16/824,790

(22) Filed: Mar. 20, 2020

Related U.S. Application Data
(60) Provisional application No. 62/831,438, filed on Apr. 9, 2019.

(51) Int. Cl. C07C 5/27; C07C 5/25; B01J 8/22; B01J 35/02; B01J 23/04; B01J 21/04; B01J 29/70

(52) U.S. Cl. CPC C07C 5/2705; C07C 5/2708; C07C 5/2518; C07C 5/2512; B01J 8/228; B01J 2208/00752; B01J 35/026; B01J 23/04; B01J 21/04; B01J 29/703; B01J 29/7026; B01J 35/023

(57) ABSTRACT

Ebullated bed reactors may be used to synthesize olefin compositions exhibiting low sediment toxicity and favorable pour points. The olefin compositions are formed by isomerizing linear alpha olefins (LAOs) into linear internal olefins (LIOs), skeletal isomerized branched olefins, or any combination thereof. Methods for preparing olefin compositions comprising LIOs and, optionally, branched olefins may comprise: providing an olefinic feed comprising one or more LAOs, and interacting the olefinic feed with a plurality of catalyst particulates in an ebullated bed reactor to form an isomerized product. The catalyst particulates are effective to isomerize the one or more LAOs into one or more of LIOs, skeletal isomerized branched olefins, or combinations thereof. The isomerized product may be incorporated in drilling fluids, particularly those intended for subsea use, due to their favorable environmental profile and low pour points. Some catalyst particulates may produce no more branching than that present in the LAOs.

### CROSS-REFERENCE OF RELATED APPLICATIONS

This application claims priority to and the benefit of U.S. Provisional Application No. 62/831,438, filed Apr. 9, 2019, the disclosure of which is incorporated by reference.

### FIELD

The present disclosure relates to olefin isomerization using ebullated bed reactors.

### BACKGROUND

Linear alpha olefins (LAOs), which also may be referred to as linear terminal olefins or linear terminal alkenes, may be synthesized by several processes starting from low molecular weight feedstock materials. The two primary processes for synthesizing LAOs are oligomerization of ethylene and byproduct isolation from the Fischer-Tropsch synthesis. LAOs may also be isolated from a petroleum refinery stream. Depending on whether LAOs originate from petroleum or are produced synthetically, a variable extent of branching may be optionally present along the main carbon chain. In addition, variable amounts of terminal and/or non-terminal branched olefins may frequently be present in an olefinic feed comprising predominantly LAOs. The amount of branched olefins in a given olefinic feed may depend upon the carbon atom count of the LAOs that are present and the process by which the LAOs were produced.

Drilling operations within an earthen formation to promote extraction of a natural resource generally utilize a fluid to accomplish functions such as, for example, removing cuttings from the wellbore, lubricating and cooling the drill bit, supporting the drill pipe, maintaining hole stability, and providing hydrostatic pressure to prevent blowouts from occurring. Fluids used in conjunction with drilling or extending a wellbore may be referred to as “drilling muds” or “drilling fluids.”

LAOs may be incorporated in drilling fluids, either as a continuous phase or as a discontinuous phase, in order to accomplish the foregoing. Thus, drilling fluids containing LAOs may be either oil-based (invert emulsion) or water-based (normal emulsion). It may be especially desirable to use LAOs, particularly C₁₆ and C₁₈ LAOs, in offshore drilling fluids because of their tendency to exhibit low sediment toxicity toward aquatic organisms and undergo biodegradation in anaerobic environments. The features of low toxicity and biodegradability offer certain advantages over less environmentally friendly hydrocarbon drilling fluids. However, C₁₆ and Cis LAOs exhibit relatively high pour point values, which may lead to problematic drilling fluid delivery for wellbores in certain locales. In particular, when substantial LAOs are present in a drilling fluid, delivery to a subsea environment may be complicated by drilling fluid solidification resulting from exposure of the drilling fluid to cold subsea temperatures that may be encountered. Some commercial LAOs of relevance to the drilling industry have pour points of +7.2° C. (C₁₆, freezing point=+3.9° C.) and +18.3° C. (C₁₈, freezing point=+17.8° C.), where pour point is measured by ASTM D97 and freezing point is measured by ASTM D1015. The pour point of a 65:35 C₁₆/C₁₈ LAO blend is +13° C.

Linear internal olefins (LIOs) may overcome the pour point difficulties associated with corresponding LAOs having the same carbon atom count. Branched olefins having a limited extent of branching, including LIOs having a limited extent of branching and skeletal isomerized olefins produced from LAOs, may similarly overcome the pour point difficulties of LAOs. Whereas LAOs feature a terminal double bond, LIOs instead include a double bond between two internal (interior) carbon atoms along the main carbon chain. LIOs may maintain the favorable environmental profile of LAOs. Compared to LAOs having the same carbon atom count, LIOs may exhibit lower pour point values and thereby maintain a liquid state at lower temperatures than is possible with the corresponding LAOs. The low pour points exhibited by LIOs may allow their delivery into wellbores located in environments that would otherwise be prohibitive due to drilling fluid solidification. Moreover, the environmental profile of LIOs may make these entities suitable for use in offshore drilling locales having stringent regulations for biodegradability and sediment toxicity, such as the Gulf of Mexico and off the coast of Brazil. Sediment toxicity specifications therein are in accordance with ASTM E-1367. Anaerobic biodegradation specifications therein are in accordance with modified ISO 11734 275-D (275 day test), and corresponding aerobic biodegradation specifications are in accordance with OECD 306. Although LIOs may be particularly desirable for offshore drilling applications, they may also be suitable for drilling in onshore locales and in other applications as well. Olefins having a limited extent of branching may offer similar features to those mentioned above when used in drilling applications.

Commercially sourced C₁₆ and C₁₈ LAOs usually contain 75-90 wt. % LAOs, in conjunction with 3-10 wt. % internal olefins (IOs) and 5-15 wt. % branched olefins (BOs). LAOs having a lower carbon atom count may contain fewer branched olefins, and similarly, those having a higher carbon atom count may tend to contain more branched olefins. LAOs may be isomerized into LIOs and/or branched olefins using a variety of heterogeneous catalysts. However, depending on the catalyst, the conditions needed to affect isomerization may be rather harsh, and additional excessive branching or cracking (breaking the LAO chain into two or more smaller chains or molecules) of the LAOs may occur under the isomerization reaction conditions. Cracking can result in undesirable product volume loss. The introduction of additional excessive branching to the main carbon chain during isomerization can sometimes be undesirable due to the tendency for the branches, particularly C₂₊ branches and/or an excessive number of branches, to decrease olefin biodegradability. Extensive random branching can be especially problematic in this regard. A limited amount of methyl branching may be less problematic or even beneficial due to the tendency of the limited branches to decrease pour points. Although a limited amount of branching in LIOs or in compositions comprising LIOs may be desirable for decreasing pour point, the isomerization catalysts and methods that are presently employed in the art oftentimes do not introduce branches controllably or predictably. At best, there is room for improvement in limiting the amount of branching produced when isomerizing LAOs into LIOs and skeletal isomerized branched olefins, particularly for improving the quality and environmental profile of drilling fluids produced therefrom.

### SUMMARY

In any embodiment, the present disclosure provides processes for synthesizing linear internal olefins (LIOs), optionally in combination with skeletal isomerized branched olefins having limited branching. The processes comprise (or consist of, or consist essentially of) providing an olefinic feed comprising one or more linear alpha olefins (LAOs), and interacting the olefinic feed with a plurality of catalyst particulates in an ebullated bed reactor to form an isomerized product comprising one or more of LIOs, skeletal isomerized branched olefins, or any combination thereof. The catalyst particulates are effective to isomerize the one or more LAOs into the one or more of LIOs, skeletal isomerized branched olefins, or any combination thereof.

In some or any embodiment, the present disclosure provides processes for synthesizing LIOs, optionally in combination with skeletal isomerized branched olefins having limited branching, in a liquid ebullated bed reactor. The processes comprise (or consist of, or consist essentially of): introducing an olefinic feed comprising one or more LAOs to an ebullated bed reactor containing a plurality of catalyst particulates, fluidizing the catalyst particulates within the ebullated bed reactor with an upward stream of an ebullating liquid, interacting the olefinic feed with the catalyst particulates under reaction conditions effective to form an isomerized product comprising one or more of LIOs, skeletal isomerized branched olefins, or any combination thereof, and removing a product stream from the ebullated bed reactor. The product stream comprises the isomerized product. The catalyst particulates are effective to isomerize the one or more LAOs into the one or more of LIOs, skeletal isomerized branched olefins, or any combination thereof.

### BRIEF DESCRIPTION OF THE DRAWING

FIG. 1 is a cross-sectional view of an illustrative ebullated bed reactor compatible for use in the present disclosure.

### DETAILED DESCRIPTION

The present disclosure relates to olefin isomerization and, more specifically, processes and apparatuses for isomerizing olefins under ebullated bed conditions. Linear internal olefins, skeletal isomerized olefins with limited branching, or a combination thereof may be produced by applying the disclosure herein.

As discussed above, there is growing demand for drilling fluids that meet new stringent environmental regulatory requirements with respect to marine sediment toxicity and biodegradability. To this end, linear alpha olefins (LAOs) can be utilized in drilling fluids, but these compounds may be problematic in certain circumstances due to their relatively high pour points. For example, when delivering a drilling fluid containing substantial LAOs to a subsea wellbore, the cold subsea temperatures may undesirably solidify the drilling fluid. LIOs or olefins having a limited amount of branching may be a suitable substitute for LAOs in drilling fluids in certain circumstances. Both LIOs and olefins having limited branching may maintain the desirably low aquatic toxicity and favorable biodegradation profiles associated with LAOs. Advantageously, moving the double bond from the terminal position in LAOs to an internal position in LIOs or olefins with limited branching may afford a desirable decrease in pour point. However, LIOs and olefins with limited branching are not straightforward to synthesize from small molecule feedstock materials. Moreover, many currently used isomerization processes for converting LAOs into LIOs and skeletal isomerized olefins are prone to promoting uncontrolled cracking or branching of the carbon chain in concert with isomerizing the position of the olefinic bond. Lower molecular weight olefins obtained from cracking may exhibit unfavorable volatility and require distillation or flashing before further use of higher molecular weight LIOs or skeletal isomerized olefins. Like LIOs, branched olefins having a limited amount of branching, including LIOs having a limited extent of branching and skeletal isomerized olefins having a limited extent of branching, may be suitably used in drilling fluids with similar advantages to those afforded by LIOs (e.g., reduced pour point and biodegradability). While olefins with a controlled/limited extent of branching may be suitably used, olefins bearing excessive branching and/or longer-chain branching (C₂₊) may be undesirable for incorporation in drilling fluids due to their lower biodegradability, particularly drilling fluids intended for offshore use.

The present disclosure describes processes and apparatuses that may be suitable for forming LIOs from LAOs, possibly with introduction of a controlled amount of branching based upon the choice of catalyst used. More specifically, the present disclosure describes ebullated bed processes and apparatuses that may be suitable for isomerizing one or more LAOs into one or more of LIOs, skeletal isomerized branched olefins having a controlled extent of branching, or any combination thereof. Different catalyst systems may be applicable for synthesizing each type of olefin or branched olefin. Catalysts suitable for synthesizing LIOs may not introduce additional branching over that already present in the LAOs undergoing isomerization. Catalysts suitable for synthesizing skeletal isomerized branched olefins, in contrast, may introduce a limited amount of branching additional to that already present in the LAOs, in addition to isomerizing the position of the olefinic bond.

Ebullated bed processes and apparatuses may be especially favorable due to their ability to fluidize highly active catalyst particulates and to allow ready catalyst replenishment to take place using fresh catalyst. Among the benefits that may be realized with the disclosure herein is that catalyst particulates suitable for ebullated bed reactors do not need to be fully formulated, unlike those used in fixed bed reactor configurations. Catalyst particulates may be significantly smaller in an ebullated bed as compared to a fixed bed (e.g., as small as 5-10 microns in size and unsupported compared to approximately 2-7 mm for fully formulated and supported catalyst particulates for fixed bed reactor configurations in which minimization of a pressure drop across the catalyst bed and crush resistance may be desired), while at the same time exhibiting much higher catalytic activity. Catalyst particulates formulated for ebullated beds also may have a much lower cost compared to the fully formulated catalysts used in fixed beds. Significantly smaller catalyst beds may also be used in ebullated bed processes, which may decrease both catalyst costs and capital equipment costs by permitting smaller reactor vessels to be used. In addition, catalyst replacement in an ebullated bed reactor may take place much more readily and with minimal system downtime compared to comparable fixed bed reactor processes. In fact, catalyst replacement may sometimes take place continuously or periodically without inducing process downtime in certain ebullated bed processes. Finally, ebullated bed processes may be conducted at lower temperatures and run at higher conversion rates than the corresponding fixed bed processes, thereby lowering the incidence of lights production (cracking). By conducting the isomerization reaction at different temperatures, the extent of branching may be controlled as well.

As referenced above, the isomerization of LAOs into LIOs may take place without the occurrence of substantial cracking or the formation of extensive new branches on the carbon chain. That is, in any embodiment, an isomerized product comprising one or more LIOs may exhibit no more branching than that present in the LAOs from which they were produced. Thus, certain processes of the present disclosure do not substantially produce additional (new) branched olefins in the course of isomerizing LAOs into LIOs. Alternately, processes of the present disclosure may be conducted such that a limited amount of short-chain branches (e.g., methyl branches) are introduced to the main carbon chain along with skeletal isomerization when producing skeletal isomerized branched olefins from a feed comprising one or more LAOs. That is, when the chosen catalyst produces skeletal isomerized branched olefins, the amount of branching may increase over that present in the LAOs but remain sufficiently limited to maintain pour point and biodegradability within a desirable range. LIOs without new branching introduced thereto may also be produced concurrently with skeletal isomerized branched olefins when using a catalyst that promotes skeletal isomerization.

In any embodiment, the ebullated bed processes of the present disclosure may be conducted with apparatuses employing an ebullating liquid. While gas-ebullated bed reactors are known in the art, particularly in hydrocracking processes, ebullated bed reactors employing an ebullating liquid are much less common, and use thereof is believed to be unknown with respect to olefin isomerization according to the disclosure herein. Liquid ebullation may provide a number of advantages compared to gas ebullation. Liquid ebullation may afford uniform mixing of reactants and catalyst particulates within a gently floating heterogeneous bed produced by a liquid olefin feed stream (ebullating liquid). No gas flow is needed in the disclosure herein, although an optional gas flow stream (e.g., N₂) may be used to sustain ebullation in some cases. As such, no additional equipment, such as a separate gas line, is needed, thereby simplifying the reactor design. The ebullating liquid used in the disclosure herein may be a reaction solvent, a feed of one or more LAOs, or any combination thereof. More typically, the ebullating liquid may comprise an upward stream of the one or more LAOs, which may comprise neat LAOs. Thus, the disclosure herein may afford greater operational simplicity compared to processes employing gas ebullation.

When compared to catalysts compatible with fixed bed catalyst systems, a catalyst bed ebullated with a liquid may allow significantly smaller catalyst particulates to be utilized. Smaller catalyst particulates may exhibit higher catalytic activity compared to larger catalyst particulates, thereby lowering catalyst usage and cost, and allow smaller reactors having lower capital equipment costs and significantly decreased mass transfer limitations to be employed. The foregoing features may also allow the isomerization temperature to be lowered, more uniform, and better controlled, thereby decreasing the likelihood of the occurrence of unwanted side products, such as excessive branching and/or cracking. Choice of the catalyst may also impact the extent of branching that occurs during a given ebullated bed process. For example, some catalysts may lead to formation of LIOs having no more branching than that present in the LAOs from which they were produced, whereas other catalysts may be chosen to form skeletal isomerized branched olefins with a limited extent of branching.

The activity of catalysts tends to decrease over time as the catalyst ages, particularly for catalysts such as zeolites. To compensate for the lower catalytic activity, the reaction temperature is often increased to maintain a desired rate of reaction. Modulation of the reaction temperature in this manner is commonly used during fixed bed reactor processes, since replacing the catalyst bed is oftentimes a very cumbersome process that may require process downtimes of several weeks or more. In an ebullated bed reactor, however, fresh catalyst is much more easily introduced, and the reaction temperature may be maintained at a substantially constant temperature to maintain a desired reaction rate (fixed or variable), thereby avoiding another difficulty of fixed bed processes that may occur as the catalyst ages. It is to be appreciated that the reaction temperature of an ebullated bed process may be raised or lowered in response to particular process needs, however. Depending on the catalyst cost and ease of recovery, the catalyst removed from the ebullated bed reactor may either be regenerated or disposed of in the disclosure herein. By avoiding progressively higher reaction temperatures necessitated by catalyst aging, ebullated bed processes may avoid temperature that may lead to cracking or excessive branching in comparable fixed bed processes.

Finally, liquid ebullated bed processes of the present disclosure may also eliminate problems associated with the start-up of fixed bed reactor systems, such as those used for the production of isomerized C₁₆/C₁₈ hydrocarbons from LAOs, including those intended for use in drilling fluids. Overactive fresh catalyst in fixed bed reactor systems may result in the production of unwanted dimers at the start of production (e.g., up to 23 wt. % at the start, when approximately 3 wt. % to 5 wt. % is desired), accompanied by production of a high percentage of branched olefins (e.g., approximately 33 wt. %, when approximately 15 wt. % or less is desired). Startup reaction inconsistency may render materials off-specification and unsuitable for formulation into drilling fluids. Up to 500 hours or longer of alignment (initial run-time) may be required with fixed bed reactor systems before on-specification products are produced. The foregoing startup difficulties usually re-occur each time the catalyst is replaced in a fixed bed reactor configuration. Thus, the continuous ebullated bed processes disclosed herein represent a particular benefit in terms of having a steady catalyst activity and forming a product with a consistent composition after an initial startup period is completed, rather than a desired product forming episodically as in fixed bed reactor systems. Although ebullated bed processes may afford a product having a more consistent composition than do related fixed bed processes, it is to be appreciated that the reaction temperature in ebullated bed processes may be raised or lowered from time to time to affect the product distribution based on particular application needs.

Accordingly, the present disclosure provides ready access to one or more of LIOs and skeletal isomerized olefins with limited branching, and olefin compositions formed therefrom that have low pour points and favorable toxicity and biodegradation profiles. In the case of the LIOs being predominantly C₁₆-C₁₈ LIOs or skeletal isomerized olefins within the same carbon count range, for example, pour points lower than −6° C. may be obtained. In some cases, pour points as low as −15° C., −17° C. or even −21° C. may be obtained. Incorporation of a limited amount of branched olefins in the olefin compositions (arising from the olefinic feed itself or formed during isomerization of one or more LAOs) may also favorably decrease the pour point while preserving biodegradability. The low pour points of LIOs and skeletal isomerized olefins formed according to the present disclosure may facilitate the formulation and use of drilling fluids in which the low pour points are maintained, along with favorable sediment toxicity and biodegradation profiles. While LAOs having certain carbon chain lengths may be favorable for producing a desired range of pour point values, it is to be appreciated that the processes disclosed herein may also be applicable to isomerizing LAOs of any desired carbon count, such as within a C₁₀-C₃₀ range, for example. LIOs or skeletal isomerized olefins having a carbon count within the C₁₆-C₁₅ or C₁₄-C₂₀ range may exhibit particular advantages when incorporated in a drilling fluid. However, LIOs having carbon counts above or below the C₁₆-C₁₈ or C₁₄-C₂₀ range may find utility in other various applications and provide particular advantages therein that are distinct from those afforded during drilling applications. In non-limiting examples, the products produced according to the disclosure herein may find direct use such as coatings (e.g., paper coatings) or undergo formulation into additional products, such as drilling fluids. Additionally, the products may be converted into specialty esters, surfactants, or isoparaffin hydrocarbons. Moreover, depending upon the range of the olefin carbon count that is present, variable amounts of branched olefins from the olefinic feed may be present, as discussed in further detail below. Intentional production of skeletal isomerized olefins having a limited extent of branching may be realized in some instances, particularly with zeolite catalysts, oftentimes through the choice of catalyst and the particular reaction conditions associated therewith. For example, zeolite catalysts that are formulated for use in an ebullated bed may afford skeletal isomerized olefins having a limited amount of branching, which may be useful in the compositions and methods disclosed herein.

Unless otherwise indicated, room temperature is 25° C.

As used in the present disclosure and claims, the singular forms “a,” “an,” and “the” shall include plural forms unless the context clearly dictates otherwise.

The term “and/or” as used in a phrase such as “A and/or B” herein is intended to include “A and B,” “A or B,” “A”, and “B.”

For the purposes of the present disclosure, the new numbering scheme for groups of the Periodic Table is used. In said numbering scheme, the groups (columns) are numbered sequentially from left to right from 1 through 18, excluding the f-block elements (lanthanides and actinides).

The term “hydrocarbon” refers to a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different numbers of carbon atoms. The term “Cₙ” refers to hydrocarbon(s) or a hydrocarbyl group having n carbon atom(s) per molecule or group, wherein n is a positive integer. Such hydrocarbon compounds may be one or more of linear, branched, cyclic, acyclic, saturated, unsaturated, aliphatic, or aromatic. When referenced with respect to an LAO or LIO, the term “Cₙ” refers to a hydrocarbyl group bearing at least one double bond.

The terms “hydrocarbyl” and “hydrocarbyl group” are used interchangeably herein. The term “hydrocarbyl group” refers to any C₁-C₁₀₀ hydrocarbon group bearing at least one unfilled valence position when removed from a parent compound. “Hydrocarbyl groups” may be optionally substituted, in which the term “optionally substituted” refers to replacement of at least one hydrogen atom or at least one carbon atom with a heteroatom or heteroatom functional group. Heteroatoms may include, but are not limited to, B, O, N, S, P, F, Cl, Br, I, Si, Pb, Ge, Sn, As, Sb, Se, and Te. Heteroatom functional groups that may be present in substituted hydrocarbyl groups include, but are not limited to, functional groups such as O, S, S═O, S(═O)₂, NO₂, F, Cl, Br, I, NR₂, OR, SeR, TeR, PR₂, AsR₂, SbR₂, SR, BR₂, SiR₃, GeR₃, SnR₃, PbR₃, where R is a hydrocarbyl group or H. Suitable hydrocarbyl groups may include alkyl, alkenyl, alkynyl, aryl, heteroaryl, cycloalkyl, heterocyclyl, and the like, any of which may be optionally substituted.

The term “alkyl” refers to a hydrocarbyl group having no unsaturated carbon-carbon bonds, and which may be optionally substituted.

The term “alkenyl” refers to a hydrocarbyl group having a carbon-carbon double bond, and which may be optionally substituted. The terms “alkene” and “olefin” are used synonymously herein. Similarly, the terms “alkenic” and “olefinic” are used synonymously herein. Unless otherwise noted, all possible geometric isomers are encompassed by these terms.

The terms “linear” and “linear hydrocarbon” refer to a hydrocarbon or hydrocarbyl group having a continuous carbon chain without side chain branching, in which the continuous carbon chain may be optionally substituted with heteroatoms or heteroatom groups.

The term “linear alpha olefin (LAO)” refers to an alkenic hydrocarbon bearing a carbon-carbon double bond at a terminal (end) carbon atom of the main carbon chain.

The term “linear internal olefin (LIO)” refers to an alkenic hydrocarbon bearing a carbon-carbon double bond at any carbon atom of the main carbon chain except for a terminal (end) carbon atom.

The terms “branch,” “branched” and “branched hydrocarbon” refer to a hydrocarbon or hydrocarbyl group having a linear main carbon chain in which a hydrocarbyl side chain extends from the linear main carbon chain. Optional heteroatom substitution may be present in the linear main carbon chain or in the hydrocarbyl side chain. The term “unbranched” refers to a straight-chain hydrocarbon or hydrocarbyl group.

The term “skeletal isomerized olefin” refers to an internal olefin having one or more side chain branches, particularly methyl and/or ethyl side chain branches. Skeletal isomerized olefins may maintain the same carbon count as the corresponding LAO from which they are produced through isomerization.

The terms “solid heterogeneous catalyst”, “heterogeneous catalyst” and “supported catalyst” refer synonymously to catalysts that are insoluble in a reaction medium in which they are placed. Such catalysts may comprise a plurality of particulates that undergo ebullation according to the disclosure herein.

The term “reactor” refers to any vessel(s) in which a chemical reaction occurs. Reactors may include both separate reactors, as well as multiple reaction zones within a single reactor apparatus and, as applicable, multiple reactions zones across multiple reactors, or multiple reactors connected in series and/or parallel.

The term “ebullated bed reactor” refers to a type of fluidized bed reactor that utilizes ebullation, or slow “floating” or “bubbling” heterogeneous systems, with dispersed catalyst particulates to achieve distribution of reactants and catalysts.

As used herein, the terms “well” and “wellbore” are used interchangeably and can include, without limitation, an oil, gas, or water production well, an injection well, or a geothermal well. As used herein, a “well” also includes at least one wellbore. A wellbore can include vertical, inclined, and/or horizontal portions, and it can be straight, curved, or bifurcated. As used herein, the term “wellbore” includes any cased portion, or any uncased, open-hole portion of the wellbore. A near-wellbore region is the subterranean material and rock of the subterranean formation surrounding the wellbore. As used herein, a “well” also includes the near-wellbore region. The near-wellbore region is generally considered to be the region within 10 feet of the wellbore, although other distances both shorter and longer are also contemplated. As used herein, the phrases “into a well” and “into a wellbore” mean and include into any portion of the well, including into the wellbore or into the near-wellbore region via the wellbore.

A portion of a wellbore may be an open-hole or cased-hole. In an open-hole wellbore portion, a tubing or drill string may be placed into the wellbore. The tubing or drill string allows fluids to be circulated in the wellbore. In a cased-hole wellbore portion, a casing is placed and cemented into the wellbore, which can also contain a tubing or drill string. The space between two cylindrical shapes is called an annulus. Examples of an annulus include, but are not limited to: the space between the wellbore and the outside of a tubing or drill string in an open-hole wellbore; the space between the wellbore and the outside of a casing in a cased-hole wellbore; and the space between the inside of a casing and the outside of a tubing or drill string in a cased-hole wellbore. A drilling fluid may be circulated in the annulus, particularly for removing a portion of the drilling fluid from the wellbore.

Accordingly, In any embodiment, processes for isomerizing LAOs into LIOs, optionally in combination with production of skeletal isomerized olefins, according to the present disclosure may comprise: providing an olefinic feed comprising one or more LAOs, and interacting the olefinic feed with a plurality of catalyst particulates in an ebullated bed reactor to form an isomerized product comprising one or more of LIOs, skeletal isomerized olefins, or any combination thereof. The catalyst particulates are effective to isomerize the one or more LAOs into the one or more of LIOs or skeletal isomerized branched olefins.

According to some embodiments, LIOs produced according to the disclosure herein may be unbranched or contain no more branching than do the one or more LAOs from which they are produced. That is, certain processes of the present disclosure do not introduce additional branches in the LIOs over those already present in branched olefins within the olefinic feed. Thus, according to some embodiments, LIOs and compositions comprising LIOs may have no branching or a controlled amount of branching may be formed based upon the amount of branching present initially in the olefinic feed. In some or other specific embodiments, substantially no cracking takes place in conjunction with isomerizing the one or more LAOs into the one or more LIOs. By “substantially no”, what is meant is that no cracking takes place, and if there is any, it is to an extent of less than 1 wt. % or less than 0.5 wt. % of the total.

Alternately, the processes for isomerizing LAOs into LIOs may take place such that a limited amount of branches are introduced during olefin isomerization. Specifically, in such processes, skeletal isomerized olefins with limited branching may be produced in combination with LIOs. Limited isomerization may comprise introducing one or two branches per molecule of skeletal isomerized olefin resulting from isomerization, wherein the one or two branches are methyl and/or ethyl branches in any embodiment. In some cases, whether a particular catalyst induces branching or not may be affected by the conditions under which the isomerization reaction is conducted.

In any embodiment, zeolite catalysts may be used to induce skeletal isomerization of LAOs into one or more olefin products with a controlled extent of branching, specifically a mixture of one or more LIOs and one or more skeletal isomerized olefins having limited branching. That is, the number of branches and length of branches are of a controlled amount. For example, the olefin products may comprise 1 to 2 branches per molecule on average, wherein the branches are methyl branches and/or ethyl branches, more typically methyl branches. Although zeolite catalysts may be used to promote controlled branching during isomerization of LAOs, such catalysts may also simply isomerize the double bond position to an internal location in some instances. Thus, isomerization of LAOs may lead to mixtures of LIOs and skeletal isomerized olefins having limited branching.

In illustrative embodiments, the olefinic feed may be comprised of any single LAO or any mixture comprising multiple LAOs, including two or more LAOs. In any embodiment, the one or more LAOs may comprise a C₁₀-C₃₀ grouping along the main carbon chain (i.e., C₁₀-C₃₀ LAOs). LIOs formed from C₁₆-C₁₈ LAOs or C₁₄-C₂₀ LAOs, in particular, may be desirable for formulation into drilling fluids, as discussed further herein. Accordingly, in more any embodiment, the one or more LAOs may comprise or consist essentially of C₁₄-C₂₀ LAOs, C₁₆-C₁₈ LAOs, C₁₆/C₁₈ LAOs, a C₁₆ LAO, a C₁₈ LAO, or any combination thereof. Illustrative LAOs that may be employed in the disclosure herein include, for example, 1-decene, 1-dodecene, 1-tetradecene, 1-hexadecene, 1-octadecene, 1-eicosene, and any variant thereof. As mentioned previously, isomerization of LAOs to form the corresponding LIOs may take place without introducing additional branching onto the carbon chain. Formation of skeletal isomerized branched olefins may take place in certain instances to introduce a limited extent of branching beyond that present in the olefinic feed.

In various embodiments, at least 50 wt. %, or at least 60 wt. %, or at least 80 wt. %, or at least 85 wt. %, or at least 95 wt. %, or at least 99 wt. % of the olefinic feed may include LAOs in the foregoing size range. Any one or more LAOs may be present in the olefinic feed. In illustrative embodiments, suitable olefinic feeds may comprise C₁₆-C₁₈ alpha olefins, such as C₁₆/C₁₈ LAO mixtures.

In any embodiment, olefinic feeds suitable for use in the disclosure herein may further comprise 5-30 wt. % branched olefins and 3-6 wt. % internal olefins. In any embodiment, suitable olefinic feeds may comprise or consist essentially of C₁₄-C₂₀ LAOs, 5-15 wt. % branched olefins, and 3-6 wt. % internal olefins. In still any embodiment, suitable olefinic feeds may comprise or consist essentially of C₁₆ LAOs or a mixture of C₁₆ and Cis LAOs, 5-15 wt. % branched olefins and 3-6 wt. % internal olefins. Other suitable olefinic feeds may comprise or consist essentially of C₁₀ LAOs or a mixture of C₁₀-C₁₄ LAOs and 5-7 wt. % branched olefins. In still any embodiment, suitable olefinic feeds may comprise or consist essentially of C₃₀ LAOs or a mixture of C₂₆-C₃₀ LAOs and 25-30 wt. % branched olefins.

In any embodiment, the olefinic feed comprising the one or more LAOs may be purified before being contacted with the catalyst particulates under ebullated bed reaction conditions. Purification may be performed at ambient temperature in order to remove moisture, stabilizers, oxygenates and/or other impurities that may impact catalyst performance and/or catalyst lifetime. Stabilizers within the LAOs may be retained in some cases. According to any embodiment, the olefinic feed may be contacted with activated alumina and zeolites, which are generally unreactive with the LAOs at room temperature, to aid in suitably purifying the olefinic feed prior to isomerization. In any embodiment, 3 Å or 4 Å zeolites, a homogeneous combination of modified activated alumina and molecular sieves, may be used to remove moisture. For example, the hybrid absorber UOP AZ-300 may be used to promote purification. Removal of oxygenates may be achieved separately with CDO-activated alumina manufactured from alumina hydroxide. Other catalyst poisons may be similarly removed or partially removed during purification of the olefinic feed. Additionally or alternately, the olefinic feed may be degassed, such as to remove dissolved gases like nitrogen, by passing the olefinic feed through a reduced pressure vessel.

According to any embodiment of the present disclosure, catalyst particulates suitable for isomerizing LAOs into LIOs and skeletal isomerized olefins with a limited extent of branching under ebullated bed reaction conditions may be zeolite catalysts, such as ZSM, MCM and USY zeolite catalysts. In any embodiment, suitable zeolite catalysts may include, for example, ZSM-11, ZSM-23, ZSM-35, ZSM-48, ZSM-57, MCM-22, MCM-41, MCM-49, USY (Zeolyst International Inc.) or other zeolite catalysts. Other suitable zeolite catalysts may include ZSM-5, ZSM-12, ZSM-23, and any combination thereof, optionally in combination with the earlier-mentioned zeolite catalysts. A microporous crystalline aluminosilicate comprising the zeolite catalyst may have a SiO₂/Al₂O₃ molar ratio of less than or equal to 100 according to any embodiment of the present disclosure, such as a SiO₂/Al₂O₃ molar ratio ranging from 5 to 100, or 10 to 80, with lower ratios providing higher catalyst activity. Zeolite technology offers several advantages in the disclosure herein, including ease of handling, high catalytic activity, good product selectivity, and facile catalyst regeneration or replacement capability. In any embodiment, the LIOs and skeletal isomerized branched olefins produced with a zeolite catalyst according to the present disclosure may feature a pour point of −21° C.

ZSM-5 is described in detail in U.S. Pat. Nos. 3,702,886 and Re. 29,948. ZSM-23 is described in U.S. Pat. No. 4,076,842. ZSM-35 is described in U.S. Pat. No. 4,016,245. ZSM-11 is described in, for example, U.S. Pat. No. 3,709,979. ZSM-12 is described in, for example, U.S. Pat. No. 4,556,477 and International Patent Application Publication 93/25475. ZSM-48 is described in, for example, U.S. Pat. No. 4,375,573. ZSM-57 is described in, for example, U.S. Pat. No. 4,973,870. MCM-41 has a hexagonal arrangement of uniformly-sized pores and is described in U.S. Pat. Nos. 5,098,684 and 5,057,296. MCM-48 has a cubic symmetry and is described in U.S. Pat. No. 5,198,203. MCM-50 has a lamellar structure and is described in U.S. Pat. No. 5,304,363. The entire contents of all the above patents are incorporated herein by reference.

Mesoporous materials having high surface Brønsted acidity, and therefore high activity, may also be employed suitably as the catalyst in the ebullated bed processes disclosed herein, particularly for isomerization reactions conducted at low temperatures. Conducting the isomerization reaction at lower temperatures may provide several benefits, such as reducing energy usage of the process and improving selectivity for desired products in the reaction mixture. Suitable mesoporous materials may have a maximum perpendicular cross-sectional pore dimension of at least 13 Angstroms, such as within the range of 13 Angstroms to 200 Angstroms, or 20 Angstroms to 60 Angstroms, or 20 Angstroms to 50 Angstroms, or 20 Angstroms to 40 Angstroms, or 20 Angstroms to 30 Angstroms, or 30 Angstroms to 60 Angstroms, or 30 Angstroms to 50 Angstroms, or 30 Angstroms to 40 Angstroms, or 25 Angstroms to 55 Angstroms.

Zeolite catalysts suitable for use in an ebullated bed may be made via a dry-spraying process to obtain particulates approximately 20 microns or larger in size or 80 microns or larger in size. Smaller particulates may also be accessible in certain instances. The catalysts consist of zeolite crystals (SiO₂/Al₂O₃), or crystals optionally attached to a high surface area support or binder, such as UOP, Versal, alumina, or others. If a binder is used, less binder may be required for the preparation of zeolite catalysts for ebullated beds (approximately 85% crystals/15% binder) versus fully formulated catalysts for fixed beds (approximately 60% crystals/40% binder).

Zeolite catalysts suitable for the disclosure herein may be supported or unsupported. When supported, the zeolite catalysts may comprise a binder or matrix material from 0 wt. % to 90 wt. % based on the weight of the isomerization catalyst, such as from 20 wt. % to 50 wt. %. Alternately, a binder or matrix material may be present at less than 10 wt. %, or less than 5 wt. %, such as from 0 to 1 wt. % based on the weight of the isomerization catalyst. Suitable binder or matrix materials may include clay, silica and/or metal oxides, which may be either naturally occurring or in the form of gelatinous precipitates or gels including mixtures of silica and metal oxides. Naturally occurring clays which may be used as a binder include those of the montmorillonite and kaolin families, such as families including the sub-bentonites and the kaolins commonly known Dixie, McNamee, Georgia or Florida clays or others in which the main mineral constituent is halloysite, kaolinite, dickite, nacrite or anauxite. Such clays can be used in their raw state as originally mined or initially subjected to calcination, acid treatment, and/or chemical modification. Suitable metal oxide binders may include silica, alumina, zirconia, titania, silica-alumina, silica-magnesia, silica-zirconia, silica-thoria, silica-beryllia, silica-titania as well as ternary compositions such as silica-alumina-thoria, silica-alumina-zirconia, silica-alumina-magnesia and silica-magnesia-zirconia.

According to any embodiment, catalyst particulates suitable for isomerizing LAOs into LIOs may be heterogeneous catalysts comprising sodium oxide (Na₂O) on a solid support, such as alumina (Al₂O₃). Other suitable solid supports may include, for example, silica or polymeric supports. Other catalyst particulates that may be suitable for isomerizing LAOs into LIOs according to the disclosure herein may include, for example, sodium or potassium carbonate (Na₂CO₃ or K₂CO₃) or sodium or potassium acetate (NaO(O═C)CH₃ or KO(O═C)CH₃) on a suitable solid support. In any embodiment, suitable catalyst particulates may include sodium carbonate, potassium carbonate, sodium acetate, potassium acetate, or any combination thereof disposed upon a solid support, such as aluminum oxide, silica or a polymer. Such catalysts may also be used without a solid support. In any embodiment, suitable catalyst particulates may contain a ratio of Na₂O:Al₂O₃ ranging from 1:10,000 to 10,000:1, or 1:1,000 to 1,000:1, or 1:500 to 500:1, or 1:250 to 250:1, or 1:100 to 100:1.

In any embodiment, catalyst particulates comprising Na₂O on alumina or a similar solid support may lead to production of LIOs that are unbranched or contain no more branching that do the one or more LAOs from which the LIOs are produced.

The catalyst particulates may be present in any suitable amount with respect to the one or more LAOs. In any embodiment, the catalyst particulates may be present in an amount ranging from 0.1% to 50% by weight of the one or more LAOs. In any embodiment, the catalyst particulates may be present in an amount ranging from 0.05% to 15% by weight of the one or more LAOs, or from 0.5% to 25% by weight of the one or more LAOs, or from 1% to 15% by weight of the one or more LAOs, or from 2% to 12% by weight of the one or more LAOs. The referenced amounts of the catalyst particulates represent the amount combined with the olefinic feed at the start of the isomerization reaction. Additional amounts of the catalyst particulates may be added, if needed, as the isomerization reaction progresses to replace spent catalyst and to maintain a desired isomerization reaction rate for converting LAOs into LIOs and/or skeletal isomerized olefins with limited branching. Spent or partially spent catalyst may be removed with a product stream. The additional amounts of the catalyst composition may be added continuously or portion-wise to the olefinic feed as the isomerization reaction occurs.

In any embodiment, suitable catalyst particulates may have a particle size ranging from 5 microns to 500 microns, or 10 microns to 200 microns, or from 80 microns to 200 microns, or from 30 microns to 100 microns, or from 30 microns to 75 microns, or from 1 microns to 10 microns. In any embodiment, the catalyst particulates may comprise a zeolite catalyst having a particle size ranging from 10 microns to 100 microns, or 30 microns to 100 microns. In other any embodiment, the catalyst particulates may comprise Na₂O on alumina and have an average particle size ranging from 80 microns to 200 microns, including average particle sizes of 100 microns or 80 microns.

In any embodiment, catalyst particulates suitable for practicing the present disclosure may have a surface area ranging from 1 m²/g to 1000 m²/g, or 5 m²/g to 900 m²/g, or 50 m²/g to 500 m²/g, or 100 m²/g to 400 m²/g. In some or any embodiment, suitable catalyst particulates may have a pore volume ranging from 0.01 cm³/g to 10 cm³/g, or 0.1 cm³/g to 5 cm³/g, or 0.5 cm³/g to 3 cm³/g, or 1 cm³/g to 2.5 cm³/g.

In any embodiment, the olefinic feed may be interacted neat with the catalyst particulates. As used herein, the term “neat” refers to a liquid phase lacking solvent. Thus, according to some embodiments of the present disclosure, olefinic feeds may be interacted with the catalyst particulates according to the present disclosure in the absence of solvent, wherein the olefinic feed is a liquid at the isomerization reaction temperature. The olefinic feed may be a liquid or a melted solid at the isomerization reaction temperature. The olefinic feed may be introduced to a reactor as an ebullating liquid, according to some embodiments. That is, according to some embodiments, the olefinic feed may be introduced to an ebullated bed reactor in a manner suitable to promote ebullation of the catalyst particulates therein, specifically as an upward stream of the ebullating liquid. Introduction of the olefinic feed as an ebullating liquid may allow the olefinic feed to be interacted neat with the catalyst particulates.

Effective distribution of the olefinic feed with catalyst particulates may be facilitated by vibration or agitation of the catalyst bed through magnetic stabilization, according to some embodiments of the present disclosure. In these systems, the solid phase may comprise a magnetizable material. An electromagnetic coil or permanent magnet may be configured to create a magnetic field inside the catalyst bed, thereby providing additional bed stability.

In any embodiments of the present disclosure, the olefinic feed may be admixed with a suitable solvent when interacting with the catalyst particulates in order to accomplish a similar result. Suitable solvents may include hydrocarbon solvents that are unreactive with the catalyst particulates, such as saturated hydrocarbon solvents. In any embodiment, an ebullating liquid being fed to the ebullated bed reactor may comprise one or more solvents that become admixed with the olefinic feed. That is, according to some embodiments, one or more solvents may be introduced to an ebullated bed reactor in a manner suitable to promote ebullation of the catalyst particulates therein. In any embodiment, a combined feed comprising one or more solvents and one or more LAOs may be introduced into the ebullated bed reactor in order to promote ebullation of the catalyst particulates therein. In any embodiment, separate feeds of the one or more solvents and the one or more LAOs may optionally be introduced to the ebullated bed reactor in order to promote ebullation of the catalyst particulates therein. If a solvent is introduced into the ebullated bed reactor, a suitable solvent separation operation may take place as the LIOs or other isomerized products are removed from the reactor.

Isomerization of one or more LAOs into one or more LIOs according to the disclosure herein may take place at a temperature ranging between 20° C. and 300° C. or 20° C. to 180° C. In more any embodiment, isomerization may take place at a temperature ranging from 100° C. to 250° C., or from 100° C. to 160° C., or from 110° C. to 150° C., or from 140° C. to 150° C., or from 20° C. to 30° C. For embodiments using a zeolite catalyst to produce a mixture of LIOs and skeletal isomerized branched olefins, isomerization may take place at a temperature ranging from 100° C. to 300° C. or 250° C. to 280° C. In any embodiment, the isomerization temperature may be 220° C. or less, which may be desirable for production of LIOs and/or skeletal isomerized olefins useful for certain drilling fluids. In any embodiment, for example, when producing isomerized olefins suitable for the production of isoparaffins, the temperature may reach approximately 280° C. or approximately 300° C. In other any embodiment, isomerization may take place at room temperature or between room temperature and 40° C. Isomerization temperatures within this lower temperature range may be particularly suitable when employing catalyst particulates comprising Na₂O on alumina or similar catalyst particulates to form substantially LIOs without additional branching over that present in the olefinic feed. In any embodiment, the isomerization reaction may be conducted initially at room temperature, and the temperature may rise during the reaction due to the exothermal nature of the isomerization process. In some or any embodiment, isomerization may take place at a temperature at which the one or more LAOs are in a liquid phase or state.

In any embodiment, isomerization may be conducted at a temperature such that substantially no additional branching occurs when isomerization of the one or more LAOs takes place. In any embodiment, the temperature may be chosen such that both a controlled amount of branching and double bond isomerization of LAOs occurs.

In any embodiment, isomerization of the one or more LAOs into LIOs and/or skeletal isomerized branched olefins may take place at atmospheric pressure. Isomerization reaction conditions that are above atmospheric pressure are also possible in any embodiment of the present disclosure.

In any embodiment, isomerization of the one or more LAOs into LIOs and/or skeletal isomerized olefins may take place under an inert atmosphere. Inert atmospheres may be favorable for maintaining activity of the catalyst composition. Suitable inert atmospheres may include, for example, helium, argon, neon, or nitrogen, for example. In some or any embodiment, isomerization of the one or more LAOs into LIOs and/or skeletal isomerized olefins may take place with substantial exclusion of water, such as through treatment of an LAO feed stream using a dryer bed employing a suitable desiccant.

In any embodiment of the present disclosure, isomerization of the one or more LAOs into LIOs and/or skeletal isomerized olefins may take place in air. Even in air (including exposure to water vapor in the air), at least some catalytic activity may be preserved.

In any embodiment, isomerization of the one or more LAOs into LIOs and/or skeletal isomerized olefins may take place over a period of time ranging from 5 minutes to 24 hours. In any embodiment, isomerization may take place over a period of time ranging from 20 minutes to 6 hours, or from 30 minutes to 4 hours, or from 30 minutes to 2 hours, or from 10 minutes to 90 minutes, or from 10 minutes to 30 minutes, or from 20 minutes to 90 minutes, or from 20 minutes to 60 minutes. Other isomerization reaction times and isomerization temperatures may be selected such that substantially all of the LAOs undergo conversion into LIOs and/or skeletal isomerized olefins. Conversion of LAOs into LIOs and skeletal isomerized olefins may be considered to be substantially complete when less than residual 5% LAOs or other alpha olefins are present. Less than 1% LAOs or other alpha olefins may be present In any embodiment.

According to any embodiment, processes for isomerizing one or more LAOs to form one or more of LIOs and skeletal isomerized olefins having limited branching may comprise: introducing an olefinic feed comprising one or more linear alpha olefins (LAOs) to an ebullated bed reactor containing a plurality of catalyst particulates effective to isomerize one or more LAOs into one or more of LIOs, skeletal isomerized olefins, or any combination thereof; fluidizing the catalyst particulates within the ebullated bed reactor with an upward stream of an ebullating liquid; interacting the olefinic feed with the catalyst particulates under reaction conditions effective to form an isomerized product comprising one or more of LIOs, skeletal isomerized branched olefins, and any combination thereof; and removing a product stream from the ebullated bed reactor, the product stream comprising the isomerized product.

Depending on the catalyst identity, the product stream may comprise one or more LIOs that are unbranched or contain no more branching than do the one or more LAOs. In any embodiment, the LAOs may undergo isomerization to produce LIOs and skeletal isomerized olefins having limited branching with branches of a controlled number and length, particularly one to two methyl branches per molecule in particular instances.

Processes for isomerizing one or more LAOs into one or more of LIOs and skeletal isomerized olefins using an upward stream of an ebullating liquid may further comprise introducing a catalyst stream comprising the catalyst particulates downwardly into the ebullated bed reactor. The catalyst particulates may comprise any substance capable of isomerizing one or more LAOs into the corresponding LIOs and/or skeletal isomerized olefins, with particular examples, including sizes and amounts thereof, being provided hereinabove.

The catalyst particulates may be introduced to the ebullated bed reactor continuously or periodically (batchwise). Any embodiment may introduce the catalyst stream continuously to the ebullated bed reactor while simultaneously or near simultaneously withdrawing a stream of at least partially spent catalyst particulates from the bottom of the ebullated bed reactor. In any embodiment, the catalyst stream may be introduced and the at least partially spent catalyst may be withdrawn from the ebullated bed reactor non-continuously (i.e., periodically or batchwise). In any embodiment, a rate of introduction of the catalyst stream and a rate of at least partially spent catalyst withdrawal may be substantially equal to one another, such that a total amount of catalyst within the ebullated bed reactor is maintained at a near-constant amount. In some or any embodiment, a rate of introduction of the catalyst stream and a rate of at least partially spent catalyst withdrawal from the ebullated bed reactor may be selected to maintain a desired rate of isomerization.

In any embodiment, the ebullating liquid may comprise the olefinic feed, such that the olefinic feed is provided as an upward stream within the ebullated bed reactor. In any embodiment, the ebullating liquid may comprise a process stream comprising one or more solvents, such that the process stream is provided as an upward stream within the ebullated bed reactor. In any embodiment, separate upward streams of the olefinic feed and the process stream may be introduced to the ebullated bed reactor. In any embodiment, a combined stream comprising the olefinic feed and the process stream may be introduced to the ebullated bed reactor. In still any embodiment, one of the olefinic feed or the process stream may be introduced to the ebullated bed reactor such that only one of the olefinic feed or the process stream promote ebullation of the catalyst particulates within the ebullated bed reactor.

As discussed above, any embodiment of the present disclosure may utilize an ebullated bed reactor having an upward stream of an ebullating liquid to promote fluidization of catalyst particulates therein. Suitable ebullated bed reactors for practicing the disclosure herein are provided hereinafter. According to some embodiments, apparatuses of the present disclosure may comprise at least one ebullated bed reactor. The apparatuses may comprise: a reactor vessel having an internal volume configured to receive a reactant stream; a first feed line configured to deliver a plurality of catalyst particulates downwardly to the internal volume; a second feed line configured to deliver an ebullating liquid upwardly to the internal volume; and a product withdrawal line configured to remove a product stream from the internal volume.

The reactant stream may be delivered to the internal volume in any suitable manner, with the manner of delivery possibly being arranged to promote ebullation within the internal volume. According to any embodiment, the apparatuses described herein may comprise a third feed line configured to deliver the reactant stream upwardly to the internal volume, thereby being arranged to promote ebullation therein. Thus, in such embodiments, the apparatuses of the present disclosure may feature both a second feed line and third feed line arranged to promote ebullation of catalyst particulates in the internal volume. In any embodiment, however, the apparatuses may comprise a third feed line that is not arranged to promote ebullation when delivering the reactant stream. In still any embodiment, the apparatuses may lack a third feed line, with the reactant stream either being delivered as the ebullating liquid from the second feed line or in combination with a solvent delivered from the second feed line.

Ebullated bed reactors may offer high quality, continuous mixing of a liquid reactant and catalyst particulates. In any embodiment, the various feeds are continuously recirculated to advantageously maintain the heterogeneous catalyst in a fluidized state within a given catalyst zone. Advantages of the ebullated bed reactors disclosed herein include temperature control, low and constant pressure drop, and controlled reaction rates. The ebullated bed reactors have the characteristics of stirred reactor-type operations, but with a hydraulically fluidized catalyst and without the difficulties associated with a fixed bed reactor system.

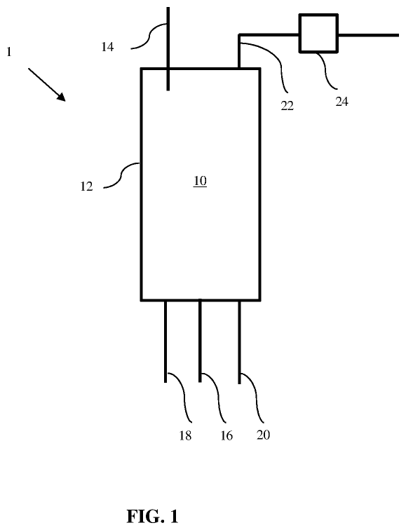

FIG. 1 shows a cross-sectional diagram of an illustrative apparatus comprising an ebullated bed reactor of the present disclosure. As shown, apparatus 1 includes reactor vessel 12 having internal volume 10 located therein. Internal volume 10 is configured to receive a reactant stream, which may comprise one or more LAOs according to the various embodiments of the present disclosure. Feed line 14 is configured to deliver a plurality of catalyst particulates downwardly to internal volume 10, such as in slurry form mixed with the reactant stream or a reaction solvent. Removal line 16 is configured to withdraw at least a portion of the catalyst particulates from the bottom of internal volume 10, such as when replacement of the catalyst particulates is desired. Optionally, delivery of catalyst particulates via feed line 14 and withdrawal of catalyst particulates via removal line 16 may take place simultaneously.

Referring still to FIG. 1, at least one source of an ebullating liquid is configured for delivery to internal volume 10. As depicted, apparatus 1 includes separate feed lines 18 and 20 to deliver an ebullating liquid (e.g., solvent) and a reactant stream (e.g., one or more LAOs) upwardly within internal volume 10. As described above, alternative configurations may omit one of feed lines 18 and 20, such that delivery of a solvent or a reactant stream does not promote ebullation in internal volume 10. In still other alternative configurations, a solvent and a reactant stream may be delivered concurrently with a single feed line 18 in order to promote ebullation in internal volume 10.

Apparatus 1 also includes removal line 22, which is configured to remove a product stream from the top of reactor 12. Alternately, removal line 22 may remove a product stream from a location other than the top of reactor 12. The product stream within removal line 22 may be further processed with inline solids separator 24, which provides a substantially solids-free product for further downstream processing. Devices for solids removal within inline solids separator 24 may include, for example, hydrocyclones, filters, settling tanks, and similar devices. Inline solids separator 24 may capture residual catalyst solids, for example, that are not removed via removal line 16.

Compositions of the present disclosure may vary to some extent depending on the particular type of catalyst particulates that are used. When the catalyst particulates comprise Na₂O on alumina, particular compositions of the present disclosure may comprise one or more C₁₄-C₂₀ LIOs and 5-15 wt. % of one or more branched olefins, wherein the branching that is present arises from the LAOs from which the compositions were produced. That is, Na₂O on alumina catalysts do not lead to substantial production of new branches in the disclosure herein. Such LIO compositions may have a pour point of −6° C. or less and be anaerobically biodegradable (ISO 11734 275-D). In any embodiment, such LIO compositions may have a pour point of −12° C. or less. In any embodiment of the present disclosure, such LIO compositions may comprise 90 wt. % or more of the one or more LIOs. In some or other any embodiment, such LIO compositions may comprise or consist essentially of C₁₆ LIOs or a mixture of C₁₆ and C₁₈ LIOs. LIO compositions formed from olefinic feeds having a higher percentage of branched olefins may display a corresponding amount of branching in the LIO compositions, but new branches are not formed according to the methods described herein.

Other compositions of the present disclosure may comprise a mixture of LIOs and skeletal isomerized branched olefins having a limited extent of branching. When the catalyst particulates comprise a zeolite, particular compositions of the present disclosure may comprise a mixture of one or more C₁₄-C₂₀ LIOs in an amount up to 80 wt. % and one or more skeletal isomerized branched olefins having a limited extent of branching in an amount up to 15 wt. %. Such LIO compositions may include additional branching arising from the LAOs from which the compositions were produced. Such LIO compositions may have a pour point of −15° C. or less or −18° C. or less and be anaerobically biodegradable (ISO 11734 275-D). In any embodiment, such LIO compositions may comprise or consist essentially of C₁₆ LIOs and skeletal isomerized olefins having a limited number of branches or a mixture of C₁₆ and C₁₈ LIOs and skeletal isomerized olefins having a limited number of branches. LIO compositions formed from olefinic feeds having a higher percentage of branched olefins may display a corresponding increase in the extent of branching compared to those formed from olefinic feeds having a lesser extent of branching.

Any of the LIO compositions disclosed herein may be formulated into a drilling fluid. The LIOs and resulting LIO compositions may have low aquatic toxicity and favorable biodegradation profiles.

Advantageously, moving the double bond from the terminal position in LAOs to an internal position in LIOs or skeletal isomerized olefins affords a desirable decrease in pour point. In the case of the LIOs being predominantly C₁₆-C₁₈ LIOs, for example, pour points lower than −6° C. may be obtained. In some cases, pour points as low as −17° C. or −21° C. may be obtained when a mixture of LIOs and skeletal isomerized branched olefins are present. Incorporation of a limited amount of branched olefins in the olefin compositions (from the olefinic feed or within skeletal isomerized olefins) may also favorably decrease the pour point while preserving biodegradability. The low pour points of LIOs, optionally in combination with skeletal isomerized olefins, may facilitate the formulation and use of drilling fluids in which the low pour points are maintained, along with favorable sediment toxicity and biodegradation profiles. It is to be appreciated that the methods disclosed herein may also be applicable to isomerizing LAOs of any desired carbon count, such as within a C₁₀-C₃₀ range, for example. LIOs and/or skeletal isomerized olefins having carbon counts above or below the C₁₆-C₁₈ or C₁₄-C₂₀ range may find utility in various applications and provide particular advantages therein that are distinct from those afforded during drilling applications.

Drilling methods of the present disclosure may feature one or more LIOs and/or skeletal isomerized olefins having a limited extent of branching, such as the various LIO compositions described hereinabove. More specifically, in various embodiments, drilling methods of the present disclosure may comprise providing a drilling fluid comprising a LIO composition, and drilling a wellbore in the presence of the drilling fluid. The LIO composition may comprise or consist essentially of one or more of C₁₄-C₂₀ LIOs that are unbranched, and 5-15 wt. % of one or more branched olefins, optionally with up to 15 wt. % skeletal isomerized olefins having a limited extent of branching may also be present. The drilling fluids may be anaerobically biodegradable (ISO 11734 275-D). According to any embodiment, the drilling fluids may meet environmental regulations for the Gulf of Mexico or coastal Brazil, for example. In some or other any embodiment, the C₁₄-C₂₀ LIOs may comprise or consist essentially of C₁₆ LIOs or a mixture of C₁₆ and C₁₈ LIOs, either alone or in combination with skeletal isomerized olefins having a limited extent of branching. In some or any embodiment, the LIO compositions within the drilling fluids may consist essentially of the one or more C₁₄-C₂₀ LIOs and the one or more branched olefins. In such drilling fluids, C₁₆/C₁₈ LIOs may constitute the majority of the LIO composition.

In any embodiment, LIO compositions suitable for use in drilling fluids of the present disclosure may feature one or two methyl groups per molecule when incorporated within a drilling fluid. Ethyl branches may be formed on rare occasions.

According to some embodiments, drilling fluids of the present disclosure may comprise at least one oil-based mud. In any embodiment, drilling fluids of the present disclosure may comprise at least one water-based mud. It is to be recognized that the term “oil-based” or “water-based” refers to the predominant continuous phase in the drilling fluid. Specifically, an oil-based mud contains a hydrocarbon or “oil” continuous (external) phase, and a water-based mud contains an aqueous or “water” continuous (external) phase. Inversion of either type of emulsion may take place during the course of a drilling operation. The LIO compositions disclosed herein may be present in either oil-based or water-based drilling fluid, optionally in further combination with other base oil components, including those described hereinafter.

Oil-based muds may include a base oil and one or more base oil additives. Numerous base oils are known in the art. Particular base oils that may be useful in the present disclosure include natural oils and synthetic oils, as well as unconventional oils (or mixtures thereof), which can be used unrefined, refined, or re-refined (the latter being known as reclaimed or reprocessed oil). Unrefined oils are those obtained directly from a natural or synthetic source and used without added purification. These include shale oil obtained directly from retorting operations, petroleum oil obtained directly from primary distillation, and ester oil obtained directly from an esterification process. Refined oils are similar to the oils discussed for unrefined oils except refined oils are subjected to one or more purification steps to improve at least one base oil property. Suitable purification processes may include solvent extraction, secondary distillation, acid extraction, base extraction, filtration, and percolation. Re-refined oils are obtained by processes analogous to refined oils but using an oil that has been previously used as a feed stock.

The LIO compositions of the present disclosure may be used in combination with any of the base oils disclosed herein, or as an alternative to any of the base oils disclosed herein.

Natural oils include animal oils, vegetable oils (castor oil and lard oil, for example), and mineral oils. Animal and vegetable oils possessing favorable thermal oxidative stability can be used. Mineral oils vary widely as to their crude source, for example, as to whether they are paraffinic, naphthenic, or mixed paraffinic-naphthenic. Oils derived from coal or shale are also useful. Natural oils vary also as to the method used for their production and purification, for example, their distillation range and whether they are straight run or cracked, hydrorefined, or solvent extracted.

Synthetic oils include hydrocarbon oil. Hydrocarbon oils include oils such as polymerized and interpolymerized olefins (e.g., polybutylenes, polypropylenes, propylene isobutylene copolymers, ethylene-olefin copolymers, and ethylene-alphaolefin copolymers, for example). PAO base stocks are commonly used as synthetic hydrocarbon oil. By way of example, PAOs derived from C₈ to C₁₄ olefins (e.g., C₈, C₁₀, C₁₂, C₁₄ olefins or mixtures thereof) may be utilized as a base oil.

Other useful fluids for use as base oils include non-conventional or unconventional base stocks that have been processed, such as catalytically, or synthesized to provide high performance characteristics. Non-conventional or unconventional base oils include one or more of a mixture of base stock(s) derived from one or more Gas-to-Liquids (GTL) materials, as well as isomerate/isodewaxate base stock(s) derived from natural wax or waxy feeds, mineral and/or non-mineral oil waxy feed stocks such as slack waxes, natural waxes, and waxy stocks such as gas oils, waxy fuels hydrocracker bottoms, waxy raffinate, hydrocrackate, thermal crackates, or other mineral oil, or even non-petroleum oil derived waxy materials such as waxy materials received from coal liquefaction or shale oil, and mixtures of such base stocks.

GTL materials are materials that are derived via one or more synthesis, combination, transformation, rearrangement, and/or degradation/deconstructive processes from gaseous carbon-containing compounds, hydrogen-containing compounds and/or elements as feed stocks such as hydrogen, carbon dioxide, carbon monoxide, water, methane, ethane, ethylene, acetylene, propane, propylene, propyne, butane, butylenes, and butynes. GTL base stocks and/or base oils are GTL materials of base oil viscosity that are generally derived from hydrocarbons; for example, waxy synthesized hydrocarbons, that are themselves derived from simpler gaseous carbon-containing compounds, hydrogen-containing compounds and/or elements as feed stocks. GTL base stock(s) and/or base oil(s) include oils boiling in the lube oil boiling range (1) separated/fractionated from synthesized GTL materials, such as, for example, by distillation and subsequently subjected to a final wax processing step, which involves either or both of a catalytic dewaxing process, or a solvent dewaxing process, to produce lube oils of reduced/low pour point; (2) synthesized wax isomerates, comprising, for example, hydrodewaxed or hydroisomerized cat and/or solvent dewaxed synthesized wax or waxy hydrocarbons; and (3) hydrodewaxed or hydroisomerized cat and/or solvent dewaxed Fischer-Tropsch (F-T) material (i.e., hydrocarbons, waxy hydrocarbons, waxes and possible analogous oxygenates), such as hydrodewaxed or hydroisomerized/followed by catalytic and/or solvent dewaxing, dewaxed F-T waxy hydrocarbons, or hydrodewaxed or hydroisomerized/followed by catalytic or solvent dewaxing, dewaxed F-T waxes, or mixtures thereof.

GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) typically have very low sulfur and nitrogen content, generally containing less than 10 ppm, and more typically less than 5 ppm of each of these elements. The sulfur and nitrogen content of GTL base stock(s) and/or base oil(s) obtained from F-T material, especially F-T wax, may be essentially nil. In addition, the absence of phosphorous and aromatics make this material especially suitable for the formulation of low SAP products.

The term GTL base stock and/or base oil and/or wax isomerate base stock and/or base oil is to be understood as embracing individual fractions of such materials of wide viscosity range as recovered in the production process, mixtures of two or more of such fractions, as well as mixtures of one or two or more low viscosity fractions with one, two or more higher viscosity fractions to produce a blend wherein the blend exhibits a target kinematic viscosity.

Some base oils may have an ester content of 50 wt. % or less, 40 wt. % or less, 30 wt. % or less, 5 wt. % or less, or 1 wt. % or less. Additionally or alternatively, some base oils may have an ester content of 40 wt. % or greater, or 50 wt. % or greater, 70 wt. % or greater, or 90 wt. % or greater.

Some base oils may have an aromatic content ranging from 0.005 wt. % to 15 wt. %, 0.01 wt. % to 10 wt. %, 0.05 wt. % to 5 wt. %, or 0.1 wt. % to 1 wt. %.

Water-based muds may include an aqueous carrier fluid, such as fresh water, salt water, sea water, or brine, optionally containing a water-miscible organic co-solvent such as an alcohol or glycol. As used herein, the term “brine” refers to a saturated aqueous salt solution. Brines may increase the weight of a drilling fluid, which can be advantageous for maintaining hydrostatic pressure in a wellbore. Illustrative weights may include a range of 5 pounds per gallon (ppg) to 20 ppg, or 10 ppg to 16 ppg. Suitable brines may include, for example, sodium chloride brines, sodium bromide brines, potassium chloride brines, potassium bromide brines, magnesium chloride brines, calcium chloride brines, and calcium bromide brines. Oil or similar hydrocarbons, including LIOs, can also be emulsified in the aqueous carrier fluid, according to some embodiments. In any embodiment, aqueous carrier fluids and water-based muds formed therefrom may be free or essentially free from oil or oil components. Suitable emulsifying agents and/or surfactants may be present, In any embodiment.

In addition to an oil-based mud or a water-based mud, drilling fluids of the present disclosure may also include further additives. The further additives may form a heterogeneous blend with a base oil or an aqueous carrier fluid. For either oil-based or water-based drilling fluids, the further additives may be dispersed in either the external phase or the internal phase of the drilling fluid. Additional additives that may be present include, but are not limited to, an acid, a base, a pH buffer, a viscosifier and/or a rheology modifier, an emulsifier, a wetting agent, a weighting agent, a fluid loss additive, a friction reducer, or any combination thereof.

Illustrative pH buffers and bases may be selected from the group consisting of magnesium oxide, potassium hydroxide, calcium oxide, and calcium hydroxide, for example. Lime is a commercially available example. The pH buffer or base can be present in a concentration in the range of 0.5 to 10.0 pounds per barrel (ppb) of the drilling fluid. The pH may range from a low of 7, 8, 9, 10, 11, or 12 to a high of 14, such as from 10 to 14.

Suitable viscosifiers and rheology modifiers may be selected from the group consisting of inorganic viscosifiers, fatty acids, including but not limited to dimer and trimer polycarboxylic fatty acids, diamines, polyamines, organophilic clays and combinations thereof. Commercially available examples of suitable viscosifiers include, but are not limited to, VG-PLUS™, available from M-I SWACO; and RHEMOD L™, TAU-MOD™, RM-63™, and combinations thereof, marketed by Halliburton Energy Services, Inc. According to some embodiments, the viscosifier and/or rheology modifier may be present in a concentration of at least 0.5 ppb of the drilling fluid. In any embodiment, the viscosifier and/or rheology modifier can also be present in a concentration of 0.5 ppb to 20 ppb, or a range of 0.5 ppb to 10 ppb, of the drilling fluid.

The drilling fluids may further include a solid lubricant, such as graphite, or a liquid friction reducer.

The drilling fluids can further include an emulsifier or a wetting agent. The emulsifier or wetting agent can be selected from the group consisting of tall oil-based fatty acid derivatives such as amides, amines, amidoamines, and imidazolines made by reactions of fatty acids and various ethanolamine compounds, vegetable oil-based derivatives, and combinations thereof. Commercially available examples of suitable emulsifiers include, but are not limited to, EZ MUL™ NT, INVERMUL™ NT, LE SUPERMUL™, and combinations thereof, marketed by Halliburton Energy Services, Inc., and MEGAMUL™, VERSAMUL™, VERSACOAT™, marketed by MI-SWACO. Commercially available examples of suitable wetting agents include, but are not limited to, DRILLTREAT™, OMC™, marketed by Halliburton Energy Services, Inc., and VERSAWET™, marketed by MI-SWACO. According to some embodiments, the emulsifier or wetting agent may be present in at least a sufficient concentration such that the drilling fluids maintain a stable emulsion or an invert emulsion. According to any embodiment, the emulsifier or wetting agent may be present in a concentration of at least 1 ppb of the drilling fluid. The emulsifier or wetting agent can also be present in a concentration in the range of 1 ppb to 20 ppb of the drilling fluid.

The drilling fluids can further include a weighting agent. In any embodiment, the weighting agent can be selected from the group consisting of barite, hematite, manganese tetroxide, calcium carbonate, and combinations thereof. Commercially available examples of suitable weighting agents include, but are not limited to, BAROID™, BARACARB™ BARODENSE™, and combinations thereof, marketed by Halliburton Energy Services, Inc. and MICROMAX™, marketed by Elkem. According to some embodiments, the weighting agent may be present in a concentration of at least 10 ppb of the drilling fluid. The weighting agent can also be present in a concentration in the range of 10 ppb to 1000 ppb, such as 10 ppb-800 ppb, of the drilling fluid.

The drilling fluids can further include a fluid loss additive. In any embodiment, the fluid loss additive can be selected from the group consisting of oleophilic polymers, including crosslinked oleophilic polymers and particulates. Commercially available examples of suitable fluid loss additives include, but are not limited to, VERSATROL™, available from M-I SWACO; and N-DRIL™ HT PLUS and ADAPTA™, marketed by Halliburton Energy Services, Inc. The fluid loss additive can also be present in a concentration in the range of 0.5 ppb to 10 ppb of the drilling fluid.

The drilling fluids can further include an ester additive. The ester additive can be present in a concentration in the range of 1 wt. % to 20 wt. %.

The drilling fluids may also optionally include one or more metal salts, MX′_{y}, where M is a Group 1 or Group 2 metal, X′ is a halogen, and y is 1 to 2. Exemplary metal salts include, NaCl, KCl, CaCl₂, MgCl₂, and the like. The total amount of such salts in the drilling fluids may range between 10 wt. % to 35 wt. % in the water phase.

Water may also be present in oil-based drilling fluids at any convenient concentration, typically at a relatively low concentration, such as 0.5 wt. % to 20 wt. %, 0.5 wt. % to 15 wt. %, 0.5 wt. % to 12.5 wt. %, 0.5 wt. % to 10 wt. %, 0.5 wt. % to 7.5 wt. %, 0.5 wt. % to 5 wt. %, 0.5 wt. % to 2.5 wt. %, 0.5 wt. % to 1 wt. %, 1 wt. % to 10 wt. %, 1 wt. % to 7.5 wt. %, 1 wt. % to 5 wt. %, 1 wt. % to 2.5 wt. %, 2.5 wt. % to 10 wt. %, 2.5 wt. % to 7.5 wt. %, 2.5 wt. % to 5 wt. %, 5 wt. % to 10 wt. %, or 5 wt. % to 7.5 wt. %.

The operation of drilling a wellbore in the presence of a drilling fluid may include creating a wellbore de novo or extending an existing wellbore. In any embodiment, a first drilling fluid may be used for drilling a first portion of the wellbore and a second drilling fluid may be used for drilling a second portion of the wellbore. For example, different drilling fluids may be used as the wellbore is extended and the subterranean conditions change. The drilling fluids of the present disclosure containing one or more LIOs and/or skeletal isomerized olefins having limited branching may be used at any point of a drilling operation. In any embodiment, a single drilling fluid may be used for drilling both the first and second portions of a wellbore.

According to any embodiment of the present disclosure, a wellbore may be located in a subsea or similar underwater environment. Thus, in various embodiments of the present disclosure, drilling fluids comprising one or more LIOs may be used to create or extend a wellbore in a subsea or similar underwater environment.

Drilling operations may include any number of additional optional steps. In any embodiment, drilling operations may further include a step of removing at least a portion of the drilling fluid from the wellbore after introduction thereof. Drill cuttings (spoils) may also be removed from the wellbore in this process. Some drilling operations may include one or more of the following optional steps: mounting and cementing of well pipes; mounting a blowout preventer or lubricator in the top of the well; drilling, at a distance from a first well, a second well against a section of the first well to the effect that the second well achieves operational contact with the first well; mounting and cementing of well pipes in the second well; mounting a blowout preventer or lubricator in the top of the second well; whereafter the drilling from one of the first or second well continues down into the reservoir and the other well which is not drilled to the reservoir is filled wholly or partially with a fluid and a drilling tool is placed in the other well and the other well is subsequently closed so that the other well can be accessed at a later point in time, and that the tool is left in the other well so that this tool can establish a connection to the one of the first or second wells into which the drilling continued. Drilling methods of the present disclosure may therefore further include one or more steps of advancing a downhole tool in the wellbore. Suitable wellbore tools are not considered to be particularly limited and will be familiar to one having ordinary skill in the art.

Still other possible steps in a drilling operation may include one or more of the following: calculating a desired path for a well of interest relative to a reference well; measuring a position of the well of interest relative to the reference well at a location along the wellbore; calculating an actual path of the well of interest based at least in part on the measured position of the well of interest relative to the at least one reference well; comparing the actual path of the at least one well of interest to the desired path of the well of interest; and adjusting a drilling system to modify the actual path of the well of interest based at least in part on a deviation between the actual path of the well of interest and the desired path of the well of interest.

Embodiments disclosed herein include:

A. Methods for isomerizing LAOs in an ebullated bed reactor. The methods comprise (or consist of, or consist essentially of): providing an olefinic feed comprising one or more linear alpha olefins (LAOs); and interacting the olefinic feed with a plurality of catalyst particulates in an ebullated bed reactor to form an isomerized product comprising one or more of linear internal olefins (LIOs), skeletal isomerized branched olefins, or any combination thereof, the catalyst particulates being effective to isomerize the one or more LAOs into the one or more of LIOs or skeletal isomerized branched olefins.

B. Methods for isomerizing LAOs in a liquid ebullated bed reactor. The methods comprise (or consist of, or consist essentially of): introducing an olefinic feed comprising one or more linear alpha olefins (LAOs) to an ebullated bed reactor containing a plurality of catalyst particulates, the catalyst particulates being effective to isomerize the one or more LAOs into one or more of linear internal olefins (LIOs), skeletal isomerized branched olefins, or any combination thereof; fluidizing the catalyst particulates within the ebullated bed reactor with an upward stream of an ebullating liquid; interacting the olefinic feed with the catalyst particulates under reaction conditions effective to form an isomerized product comprising one or more of linear internal olefins (LIOs), skeletal isomerized branched olefins, or any combination thereof; and removing a product stream from the ebullated bed reactor, the product stream comprising the isomerized product.

Embodiments A and B may have one or more of the following additional elements in any combination:

Element 1: wherein the catalyst particulates comprise a zeolite catalyst.

Element 2: wherein the zeolite catalyst is selected from the group consisting of ZSM-11, ZSM-23, ZSM-35, ZSM-48, ZSM-57, MCM-22, MCM-41, MCM-49, and USY.

Element 3: wherein the ebullated bed reactor is ebullated with an ebullating liquid.

Element 4: wherein the ebullating liquid comprises the olefinic feed or a process stream comprising one or more solvents.

Element 5: wherein the olefinic feed is interacted neat with the catalyst particulates.

Element 6: wherein the olefinic feed comprises C₁₀-C₃₀ LAOs.

Element 7: wherein the olefinic feed comprises a C₁₆ LAO, a C₁₈ LAO, or any combination thereof.

Element 8: wherein the olefinic feed comprises C₁₄-C₂₀ LAOs.

Element 9: wherein the olefinic feed consists essentially of C₁₄-C₂₀ LAOs, 5-15 wt. % branched olefins, and 3-6 wt. % internal olefins.

Element 10: wherein the olefinic feed consists essentially of C₁₆ LAOs or a mixture of C₁₆ and C₁₈ LAOs, 5-15 wt. % branched olefins, and 3-6 wt. % internal olefins.

Element 11: wherein substantially no cracking occurs upon isomerizing the one or more LAOs to form the one or more of LIOs or skeletal isomerized branched olefins.

Element 12: wherein isomerization takes place at temperature ranging from 100° C. to 160° C.

Element 13: wherein the isomerized product has a pour point of −12° C. or lower.

Element 14: wherein the catalyst particulates have a particle size ranging from 20 microns to 100 microns.

Element 15: wherein the process further comprises: introducing a catalyst stream downwardly into the ebullated bed reactor, the catalyst stream comprising the catalyst particulates.

Element 16: wherein the catalyst stream is continuously introduced into the ebullated bed reactor.

Element 17: wherein the catalyst particulates comprise Na₂O on alumina.

Element 18: wherein the one or more LIOs are unbranched or contain no more branching than do the one or more LAOs.

By way of non-limiting example, exemplary combinations applicable to A include 1 and 2; 1 and 3; 1 and 4; 1 and 5; 1 and any one of 6-10; 1 and 11; 1 and 12; 1 and 13; 1 and 14; 1 and 17; 1, 17 and 18; 3 and 4; 3 and 5; 3 and any one of 6-10; 3 and 11; 3 and 12; 3 and 13; 3 and 14; 3 and 17; 3, 17 and 18; 4 and 5; 4 and any one of 6-10; 4 and 11; 4 and 12; 4 and 13; 4 and 14; 4 and 17; 4, 17 and 18; 5 and any one of 6-10; 5 and 11; 5 and 12; 5 and 13; 5 and 14; 5 and 17; 5, 17 and 18; any one of 6-10 and 11; any one of 6-10 and 12; any one of 6-10 and 13; any one of 6-10 and 14; 6 and 17; 6, 17 and 18; 11 and 12; 11 and 13; 11 and 14; 1 and 17; 11, 17 and 18; 12 and 13; 12 and 14; 12 and 17; 12, 17 and 18; 13 and 14; 13 and 17; and 13, 17 and 18. By way of further non-limiting example, exemplary combinations applicable to B include 1 and 2; 1 and 3; 1 and 5; 1 and any one of 6-10; 1 and 11; 1 and 12; 1 and 13; land 14; land 17; 1, 17 and 18; 3 and 5; 3 and any one of 6-10; 3 and 11; 3 and 12; 3 and 13; 3 and 14; 3 and 17; 3, 17 and 18; 5 and any one of 6-10; 5 and 11; 5 and 12; 5 and 13; 5 and 14; 3 and 17; 3, 17 and 18; any one of 6-10 and 11; any one of 6-10 and 12; any one of 6-10 and 13; any one of 6-10 and 14; 6 and 17; 6, 17 and 18; 11 and 12; 11 and 13; 11 and 14; 11 and 17; 11, 17 and 18; 12 and 13; 12 and 14; 12 and 17; 12, 17 and 18; 13 and 14; 13 and 17; and 13, 17 and 18, any of which may be in further combination with element 15 or element 16. Another exemplary combination applicable to B is 15 and 16.

### EXAMPLES

All example experiments with Na₂O were performed in a glove box under N₂ to eliminate the effects of oxygen and moisture upon catalyst performance. When used, the Na₂O on alumina catalyst was in powder form having an N₂ BET surface area of 80-130 m²/g. The catalyst particles ranged from 40 microns to 75 microns in size. The catalyst contained 11-15 wt. % sodium and had a particle density of 3.14 g/cm³. Pour point values were measured using ASTM method D5950-“Standard Test Method for Pour Point of Petroleum Products (Automatic Tilt Method).”

### Example 1

ZSM-48 catalyst (SiO₂/Al₂O₃=70) having a particle size of 30 microns to 75 microns was loaded into a Parr reactor and activated/pretreated overnight (approximately 8-10 hours) with a purge of hot N₂ at 250° C., stirring at 500 rpm. An olefin feed comprising 150 g of C₁₆ LAOs was purified using AZ-300 activated alumina, and then preheated at 150° C. before introduction into the Parr reactor. The catalyst load was 2 g of catalyst to 150 g of C₁₆ LAOs. The reaction was run as a slurry of catalyst with olefin for approximately 50 minutes at 150° C. No additional catalyst support was used.

Upon completion of the reaction, the isomerized product was quickly removed from the reactor, cooled to room temperature, and then filtered. Spent catalyst showed some color change from snow white to beige-brownish. The isomerized fluid produced from the reaction was filtered to separate and remove the catalyst powder, and was then analyzed using gas chromatography (GC) and nuclear magnetic resonance spectroscopy (NMR). GC analyses were conducted by hydrogenating 1 g of the reaction product over an Ir catalyst and analyzing the paraffinic product. ¹H NMR was used to identify the relative content of unreacted LAOs with respect to other isomerized olefin products. ¹³C NMR was used to identify the relative composition of the linear and branched isomers in the isomerized product mixture. Results of the analysis showed that the product contained less than 5% LAOs, approximately 82% LIOs, and approximately 16.5% branched olefins. The pour point of the product was −18° C.

### Example 2

ZSM-48 catalyst (SiO₂/Al₂O₃=70) having a particle size of 70 microns to 100 microns was loaded into a Parr reactor. The same experimental setup, and procedure as in Example 1 was used except as described below. The catalyst load was 1 g of catalyst to 150 g of C₁₆ LAOs. The reaction was run as a slurry of catalyst with olefin for approximately 35 minutes at 150° C. No additional catalyst support was used. Results of the analysis showed that the product contained less than 35% LAOs, approximately 40% LIOs, and approximately 9% branched olefins. The pour point of the product was −12° C.

### Example 3

ZSM-48 catalyst (SiO₂/Al₂O₃=70) having a particle size of 30 microns to 40 microns was loaded into a Parr reactor. The same experimental setup, and procedure as in Example 1 was used except as described below. The catalyst load was 0.5 g of catalyst to 150 g of C₁₆ LAOs. The reaction was run as a slurry of catalyst with olefin for approximately 35 minutes at 150° C. No additional catalyst support was used. Results of the analysis showed that the product contained less than ˜8% LAOs, approximately 77% LIOs, and approximately 13% branched olefins. The pour point of the product was −21° C.

### Example 4

ZSM-57 (SiO₂/Al₂O₃=45) catalyst having a particle size of 30 microns to 75 microns was loaded into a 30 milliliter scintillation vial and activated/pretreated for approximately 2 hours with a purge of hot N₂ at 130° C., stirring at 500 rpm. An olefin feed of C₁₆ LAOs (10 g) was purified using AZ-300 activated alumina. The catalyst load was 1 g of catalyst to 10 g of C₁₆ LAOs. The C₁₆ LAO feed was added to the heated vial via syringe. The reaction was run as a slurry of catalyst with olefin for approximately 4 hours at 130° C. No additional catalyst support was used.

Upon completion of the reaction, the isomerized product was quickly removed from the reactor, cooled to room temperature, and then filtered. Spent catalyst showed some color change from snow white to beige-brownish. The isomerized fluid produced from the reaction was filtered to separate and remove the catalyst powder, and was then analyzed using GC and NMR as described in Example 1. Results of the analysis showed that the product contained approximately 1% LAOs, approximately 86% LIOs, and approximately 13% BOs.

### Example 5

ZSM-57 (SiO₂/Al₂O₃=45) catalyst having a particle size of 30 microns to 75 microns was loaded into a 30 milliliter scintillation vial and was activated/pretreated for approximately 2 hours with a purge of hot N₂ at 110° C., stirring at 500 rpm. An olefin feed of C₁₆ LAOs (10 g) was purified using AZ-300 activated alumina. The catalyst load was 1 g of catalyst to 10 g of C₁₆ LAOs. The C₁₆ LAO feed was added to the heated vial via syringe. The reaction was run as a slurry of catalyst with olefin for approximately 4 hours at 110° C. No additional catalyst support was used.

Upon completion of the reaction, the isomerized product was quickly removed from the reactor, cooled to room temperature, and then filtered. Spent catalyst showed some color change from snow white to beige-brownish. The isomerized fluid produced from the reaction was filtered to separate and remove the catalyst powder, and was then analyzed using to gas chromatography and NMR as described in Example 1. Results of the analysis showed that the product contained approximately 22% LAOs, approximately 65% LIOs, and approximately 12% BOs.

### Example 6

A sample of 10 g of 99.5 wt. % C₁₆ LAOs (Sigma-Aldrich) was weighed into a 30 mL scintillation vial and combined with 1 g of the Na₂O/Al₂O₃ catalyst (10 wt. % catalyst loading with respect to olefinic feed). The catalyst was added to the C₁₆ LAOs over a period of 5 seconds. The reaction mixture was stirred as a slurry at 500 rpm at 21° C. over 1 hour. After 1 hour, 1 mL of water was added to the reaction mixture to quench the catalyst.

After quenching, the reaction mixture was filtered from the catalyst and analyzed using ¹H NMR. The analyses showed essentially complete (>95%) conversion of the C₁₆ LAOs into the corresponding LIOs with no introduction of branching to the carbon chain. Less than 1 wt. % of LAOs remained in the reaction product.

The pour point of the isomerized product was −15.2° C.

### Example 7

A sample of 300 g of 92 wt. % C₁₆ LAOs containing ˜6% branched C₁₆ vinylidenes was weighed into a 500 mL roundbottom flask and combined with 5 g of the Na₂O/Al₂O₃ catalyst (1.67 wt. % catalyst loading with respect to olefinic feed). The catalyst was added to the C₁₆ LAOs over a period of 5 minutes. The reaction mixture was stirred as a slurry at 500 rpm at 21° C. over 1 hour. After 1 hour, 5 mL of water was added to the reaction mixture to quench the catalyst.

After quenching, the reaction mixture was filtered from the catalyst and analyzed using gas chromatography (GC) as described in Example 1. The GC analyses showed 0% trace LAOs, 94% linear internal olefins (analyzed as the corresponding paraffins), and 6% branched olefins (analyzed as the corresponding paraffins), which is consistent with the amount of branching in the olefinic feed.

The pour point of the isomerized product was −5.5° C.

### Example 8

A sample of 300 g of 92 wt. % C₁₆ LAOs containing ˜6% branched C₁₆ vinylidenes was weighed into a 500 mL roundbottom flask and combined with 10 g of the Na₂O/Al₂O₃ catalyst (3.33 wt. % catalyst loading with respect to olefinic feed). The catalyst was added to the C₁₆ LAOs over a period of 5 minutes. The reaction mixture was stirred as a slurry at 500 rpm at 21° C. over 1 hour. After 1 hour, 10 mL of water was added to the reaction mixture to quench the catalyst.

After quenching, the reaction mixture was filtered from the spent catalyst and was analyzed using GC as described in Example 1. The analyses showed essentially complete conversion of the C₁₆ LAOs into the corresponding LIOs without introducing additional branching to the carbon chain.

The pour point of the isomerized product was −9.5° C. The lower pour point of Example 6 compared to Example 5 is believed to be due to a greater cis/trans isomer ratio and/or additional internal shift of the olefinic bond when using a higher amount of catalyst.

### Example 9

A sample of 300 g of C₁₆/C₁₈ LAOs (65 wt. % C₁₆ LAOs of total LAOs, 35 wt. % C₁₈ LAOs of total LAOs, about 90 wt. % LAOs total, with the remainder being 3-6 wt. % internal olefins and 5-10 wt. % branched olefins) was weighed into a 500 mL roundbottom flask and combined with 5 g of the Na₂O/Al₂O₃ catalyst (1.67 wt. % catalyst loading with respect to olefinic feed). The catalyst was added to the C₁₆/C₁₈ LAOs over a period of 5 minutes. The reaction mixture was stirred as a slurry at 500 rpm at 40° C. over 1 hour. After 1 hour, 5 mL of water was added to the reaction mixture to quench the catalyst.

After quenching, the reaction mixture was filtered from the spent catalyst and was analyzed using GC as described in Example 1. The analyses showed essentially complete conversion of the C₁₆/C₁₈ LAOs into the corresponding LIOs without introducing additional branching to the carbon chain.

The pour point of the isomerized product was −2.6° C.

### Example 10

A sample of 300 g of C₁₆/C₁₈ LAOs (65 wt. % C₁₆ LAOs of total LAOs, 35 wt. % C₁₈ LAOs of total LAOs, about 90 wt. % LAOs total, with the remainder being 3-6 wt. % internal olefins and 5-10 wt. % branched olefins) was weighed into a 500 mL roundbottom flask and combined with 10 g of the Na₂O/Al₂O₃ catalyst (3.33 wt. % catalyst loading with respect to olefinic feed). The catalyst was added to the C₁₆/C₁₈ LAOs over a period of 5 minutes. The reaction mixture was stirred as a slurry at 500 rpm at 40° C. for over 2 hours. After 2 hours, 10 mL of water was added to the reaction mixture to quench the catalyst.

After quenching, the reaction mixture was filtered from the catalyst and analyzed using GC as described in Example 1. The analyses showed essentially complete conversion of the C₁₆/C₁₈ LAOs into the corresponding LIOs without introducing additional branching to the carbon chain.

The pour point of the isomerized product was −8.8° C.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein.

The term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

1. A process comprising:
providing an olefinic feed comprising one or more linear alpha olefins (LAOs); and
interacting the olefinic feed with a plurality of catalyst particulates in an ebullated bed reactor to form an isomerized product comprising one or more of linear internal olefins (LIOs), skeletal isomerized branched olefins, or any combination thereof, the catalyst particulates being effective to isomerize the one or more LAOs into the one or more of LIOs or skeletal isomerized branched olefins.

2. The process of claim 1, wherein the catalyst particulates comprise a zeolite catalyst.

3. The process of claim 2, wherein the zeolite catalyst is selected from the group consisting of ZSM-11, ZSM-23, ZSM-35, ZSM-48, ZSM-57, MCM-22, MCM-41, MCM-49, and USY.

4. The process of claim 1, wherein the ebullated bed reactor is ebullated with an ebullating liquid.

5. The process of claim 4, wherein the ebullating liquid comprises the olefinic feed or a process stream comprising one or more solvents.

6. The process of claim 4, wherein the olefinic feed is interacted neat with the catalyst particulates.

7. The process of claim 1, wherein the olefinic feed comprises C₁₀-C₃₀ LAOs.

8. The process of claim 1, wherein the olefinic feed comprises a C₁₆ LAO, a C₁₈ LAO, or any combination thereof.

9. The process of claim 1, wherein the olefinic feed comprises C₁₄-C₂₀ LAOs.

10. The process of claim 1, wherein the olefinic feed consists essentially of C₁₄-C₂₀ LAOs, 5-15 wt. % branched olefins, and 3-6 wt. % internal olefins.

11. The process of claim 1, wherein the olefinic feed consists essentially of C₁₆ LAOs or a mixture of C₁₆ and C₁₈ LAOs, 5-15 wt. % branched olefins, and 3-6 wt. % internal olefins.

12. The process of claim 1, wherein substantially no cracking occurs upon isomerizing the one or more LAOs to form the one or more LIOs or the skeletal isomerized branched olefins.

13. The process of claim 1, wherein isomerization takes place at temperature ranging from 100° C. to 250° C.

14. The process of claim 1, wherein the isomerized product has a pour point of −12° C. or lower.

15. The process of claim 1, wherein the catalyst particulates have a particle size ranging from 20 microns to 100 microns.

16. The process of claim 1, wherein the catalyst particulates comprise Na₂O on alumina.

17. The process of claim 16, wherein the one or more LIOs are unbranched or contain no more branching than do the one or more LAOs.

18. A process comprising:
introducing an olefinic feed comprising one or more linear alpha olefins (LAOs) to an ebullated bed reactor containing a plurality of catalyst particulates, the catalyst particulates being effective to isomerize the one or more LAOs into one or more of linear internal olefins (LIOs), skeletal isomerized branched olefins, or any combination thereof;
fluidizing the catalyst particulates within the ebullated bed reactor with an upward stream of an ebullating liquid;
interacting the olefinic feed with the catalyst particulates under reaction conditions effective to form an isomerized product comprising one or more of linear internal olefins (LIOs), skeletal isomerized branched olefins, or any combination thereof; and
removing a product stream from the ebullated bed reactor, the product stream comprising the isomerized product.

19. The process of claim 18, further comprising introducing a catalyst stream downwardly into the ebullated bed reactor, the catalyst stream comprising the catalyst particulates.

20. The process of claim 19, wherein the catalyst stream is continuously introduced into the ebullated bed reactor.

21. The process of claim 18, wherein the catalyst particulates comprise a zeolite catalyst.

22. The process of claim 21, wherein the zeolite catalyst is selected from the group consisting of ZSM-11, ZSM-23, ZSM-35, ZSM-48, ZSM-57, MCM-22, MCM-41, MCM-49, and USY.

23. The process of claim 18, wherein the catalyst particulates have a particle size ranging from 20 microns to 100 microns.

24. The process of claim 18, wherein the ebullating liquid comprises the olefinic feed or a process stream comprising one or more solvents.

25. The process of claim 18, wherein the isomerized product is formed at a temperature ranging from 100° C. to 250° C.

26. The process of claim 18, wherein the catalyst particulates comprise Na₂O on alumina.

27. The process of claim 26, wherein the one or more LIOs are unbranched or contain no more branching than do the one or more LAOs.
